(19) United States
(12) Patent Application Publication
(10) Pub. No.: US 2024/0238029 A1
(43) Pub. Date: Jul. 18, 2024

(54) CRYOGENIC CATHETERS

(71) Applicant: Medtronic Ireland Manufacturing Unlimited Company, Dublin (IE)

(72) Inventors: Carlos H. Lima, Santa Rosa, CA (US); Brian J. Castelli, Santa Rosa, CA (US); Manoj Kumar Singh, Santa Rosa, CA (US)

(21) Appl. No.: 18/577,340

(22) PCT Filed: Aug. 26, 2022
(86) PCT No.: PCT/EP2022/073821

§ 371 Date: Jan. 8, 2024

Related U.S. Application Data
(60) Provisional application No. 63/238,904, filed on Aug. 31, 2021.

(51) Int. Cl. A61B 18/02; A61B 18/00

(52) U.S. Cl. CPC A61B 18/02; A61B 2018/00166; A61B 2018/00226; A61B 2018/00511; A61B 2018/00577; A61B 2018/0212; A61B 2018/0262

(57) ABSTRACT

A medical device includes an elongate shaft having a proximal end portion, a distal end portion, and a defining a lumen therebetween. The distal end portion defines al least one slot. An expandable member is disposed, within the lumen, the expandable member being configured to protrude outward from the at least one slot when expanded with cryogenic fluid.

### FIELD

The present technology is generally related to cryogenic catheters, and in particular, cryogenic balloon catheters for targeted ablation therapy.

### BACKGROUND

Cryotherapy is a useful treatment modality for many types of medical procedures. In some cases, it is desirable to administer cryotherapy from within a patient's body, such as from within a body lumen. Internal administration of cryotherapy can be advantageous, for example, in at least some neuromodulation procedures. These procedures can include percutaneously introducing a cryotherapeutic element into a patient and then advancing a catheter shaft carrying the cryotherapeutic element along an intravascular path to a suitable treatment location. Once positioned at the treatment location, the cryotherapeutic element can be cooled to modulate nearby nerves. The cooling caused by the cryotherapeutic element, for example, can reduce undesirable local or systemic sympathetic neural activity and thereby achieve various therapeutic benefits.

### SUMMARY

The techniques of this disclosure generally relate to cryogenic catheters, and in particular, cryogenic balloon catheters for targeted ablation therapy.

Some aspects of the present disclosure involve the recognition that, in cryogenic catheters that include a balloon, inflation of the balloon may occlude blood flow, and that occluding the renal artery may be undesirable. For example, occluding the renal artery could lead to a temporary increase in blood pressure, among other effects. Some aspects of the present disclosure relate to cryogenic catheters that allow blood flow while applying a cryotherapeutic treatment.

In one aspect, a medical device includes an elongate shaft having a proximal end portion, a distal end portion, and defining a lumen therebetween. The distal end portion defines at least one slot. An expandable member is disposed within the lumen, the expandable member being configured to protrude outward from the at least one slot when expanded with cryogenic fluid.

In another aspect of this embodiment, the expandable member is an inflatable balloon.

In another aspect of this embodiment, the distal end portion is biased to a linear configuration.

In another aspect of this embodiment, the distal end portion is biased to a spiral configuration.

In another aspect of this embodiment, the at least one slot includes a plurality of slots, and wherein the plurality of slots is longitudinally spaced along the distal end portion.

In another aspect of this embodiment, the plurality of slots is disposed at a same radially position along the distal end portion.

In another aspect of this embodiment, each of the plurality of slots is a same size and evenly spaced from each other along the distal end portion.

In another aspect of this embodiment, the device further includes at least one additional distal end portion defining at least one slot through which the expandable member or an additional expandable member protrudes outward when expanded with cryogenic fluid.

In another aspect of this embodiment, the at least one slot is an elongated slot extending along a longitudinal portion of the distal end portion.

In another aspect of this embodiment, the device further includes a stop disposed within the lumen within the distal end portion, the stop being configured to restrict longitudinal expansion of the expandable member.

In one aspect, a medical device includes a first elongate shaft having a first proximal end portion and a first distal end portion and defining a first lumen therethrough. The first distal end portion defines at least one first slot. A first expandable member is disposed within the first lumen, the first expandable member being configured to protrude outward from the at least one first slot when expanded. A second elongate shaft having a second proximal end portion and a second distal end portion is included and defines a second lumen therethrough. The second distal end portion defines at least one second slot. A second expandable member is disposed within the second lumen, the second expandable member being configured to protrude outward from the at least one second slot when expanded. The first and a second elongate shafts are disposed within a guide catheter.

In another aspect of this embodiment, the first and second expandable members are inflatable balloons.

In another aspect of this embodiment, the first and a second distal end portions are curved to define a spiral configuration.

In another aspect of this embodiment, the at least one first and second slots includes a plurality of first and second slots respectively, and wherein the plurality of first and second slots is longitudinally spaced along the respective first and second distal end portions.

In another aspect of this embodiment, the plurality of first and second slots is disposed at a same radial position along the respective first and a second distal end portions.

In another aspect of this embodiment, each of the plurality of first and second slots is a same size.

In another aspect of this embodiment, the plurality of first and second slots is evenly spaced along the respective first and second distal end portions.

In another aspect of this embodiment, the at least one first and second slots is an elongated slot extending along a longitudinal portion of the respective first and second distal end portions.

In another aspect of this embodiment, the device further includes a first stop disposed within the first lumen within the first distal end portion, the first stop being configured to restrict longitudinal expansion of the first expandable member.

In one aspect, a medical device includes an elongate shaft having a proximal end portion, a distal end portion, and defining a lumen therebetween. The distal end portion defines a plurality of longitudinally and evenly spaced slots. An inflatable balloon is disposed within the lumen, the inflatable balloon being configured to protrude outward from the plurality of slots when inflated with a cryogenic fluid. A stop is disposed within the lumen the stop being configured to restrict longitudinal inflation of the inflatable member.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a system view of an exemplary cryogenic medical system and medical device constructed in accordance with the principles of the present application;

FIG. 2 is a perspective view of the distal end portion of the medical device shown in FIG. 1;

FIG. 3 is a perspective view of the distal end portion of another embodiment of the medical device shown in FIG. 1;

FIGS. 4A-4B show perspective views of the distal end portion of another embodiment of the medical device shown in FIG. 1; and

FIG. 5 is a side view and a front of the distal end portion of another embodiment of the medical device shown in FIG. 1.

### DETAILED DESCRIPTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

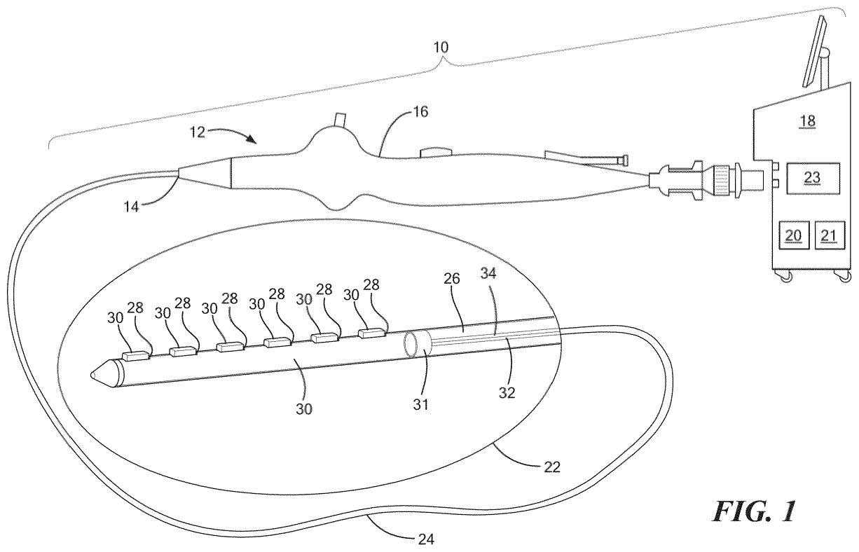

Referring now to the drawings in which like reference designators refer to like elements, there is shown in FIG. 1 an exemplary medical system constructed in accordance with the principles of the present application and designated generally as “10.” The system may include a medical device 12 including a proximal end portion 14 in fluid communication with a handle 16. The handle 16 can also include connectors that are matable directly to a cryogenic fluid supply/exhaust and console or indirectly by way of one or more umbilicals (not shown). In the exemplary system, the fluid supply and exhaust, as well as various control mechanisms for the system are housed with a single control unit or console 18. In one embodiment, the console 18 may include a refrigerant source 20 or a fluid supply reservoir, an exhaust chamber 21, and processing circuitry 23 having a processor and a memory configured to monitor and control the delivery and/or exhaust of refrigerant from the medical device 12. The console 18 is configured to deliver refrigerant, such as nitrous oxide (N₂O), which passes through the internal piping of the console 18 before being transferred to the medical device 12 via the umbilicals. As the refrigerant is released in the medical device 12 under vacuum, the temperature of the fluid drops because of the positive Joule-Thomson coefficient of nitrous oxide. Then the liquid evaporates as it absorbs heat from the medical device 12, which results in the freezing of portions of the device 12. The refrigerant vapor is then returned through the vacuum path via the umbilical and into the console 18, where it may be evacuated through a scavenging line or through the exhaust chamber 21. In addition to providing an exhaust function for the fluid supply of the medical device 12, the console 18 can also recover and/or recirculate the cooling fluid. As such, the system 10 may be referred to herein as a closed-loop system.

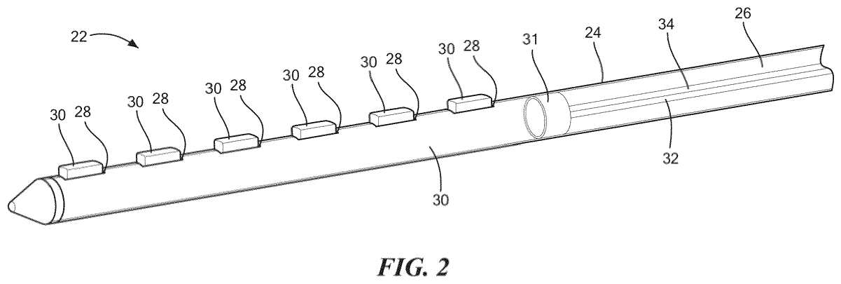

Referring now to FIGS. 1 and 2, the medical device 12 may further include a distal end portion 22 about which cryogenic energy is exchanged between the medical device 12 and a target tissue, for example, renal tissue. For example, the medical device 12 may include an elongate shaft 24 defining the proximal end portion 14 and the distal end portion 22, and which further defines a lumen 26 there through. The distal end portion 22 may define at least one slot 28 disposed along a longitudinal portion of the distal end portion 22. In some configurations, a plurality of the at least one slots 28 is included in which each of the slots 28 is longitudinally spaced from an adjacent slot 28 and may further be disposed at the same radial position as each and every other slot 28. Each of the slots 28 is sized to define an area less than a cross-sectional area of the lumen 26. In some configurations, each slot 28 is the same size and in other configurations may be differently sized. In some configurations the slots 28 define a rectangular shape, but may define any shape. Disposed within the lumen 26 is an expandable member 30 sized and configured to receive a cryogenic fluid, for example, nitrous oxide. To that end, a supply lumen 32 and an exhaust lumen 34 may be in fluid communication with the expandable member 30 to inflate and deflate, respectively, the expandable member 30. In an exemplary configuration, the expandable member 30 is compliant and when inflated with cryogenic fluid is configured to at least partially protrude outward from the at least one slot by a predetermined amount. For example, as shown in FIGS. 1 and 2, when inflated, the expandable member 30 is squeezed outward from the at least one slot 28 to provide a contact surface to cryogenically ablate tissue. In configurations in which a plurality of the at least one slots 28 are included, the expandable member 30 may protrude outward from each of the slots 28 to form multiple contact surfaces for cryogenic ablation. In some configurations, the expandable member 30 protrudes evenly from each of the plurality of the at least one slots 28. The elongate shaft 24 may be, for example, a sized 4-7F hypotube to constrain the expandable member 30 therein.

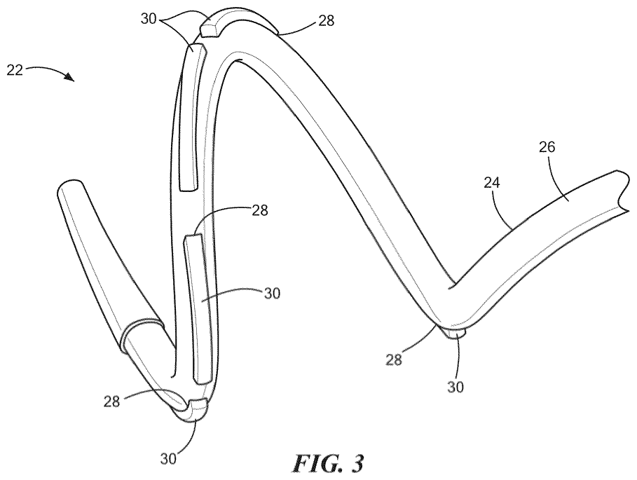

In some configurations, the distal end portion 22 of the elongate shaft 24 can have a linear or straight shape in absence of a deforming force. In some configurations, for example as shown in FIG. 3, the distal end portion 22 can have a shape of a coil, helix, or spiral in absence of a deforming force. In some configurations, the distal end portion 22 can be flexible. For example, the distal end portion 22 can be straight when delivered (e.g., to facilitate delivery) and deploy to a coil, helix, or spiral shape (e.g., for applying a treatment to tissue). The distal end portion 22 can be biased to a coil, helix, or spiral shape, which can be predefined, or the elongate shaft 24 may be torqueable or otherwise deformable (e.g., by inflation of the expandable member 30 or by retraction of a pull wire attached at or near the distal end of the distal end portion 22) in situ to a spiral, helix, or coil shape. In some configurations in which the distal end portion 22 is biased to a deployed shape, the distal end portion 22 can be straightened from the deployed shape for delivery, for example, by advancing a guidewire or control member through the elongate shaft 24 and the distal end portion 22 or by constraining the distal end portion 22 within a sheath or catheter (e.g., a guide catheter). In some configurations, the distal end portion 22 can be biased to be straight or linear absent deforming force. In some configurations, the distal end portion 22 of the elongate shaft 24 may be rigid, that is to say that the distal portion is substantially unbendable, while in other configurations the distal end portion 22 of the elongate shaft 24 may be flexible.

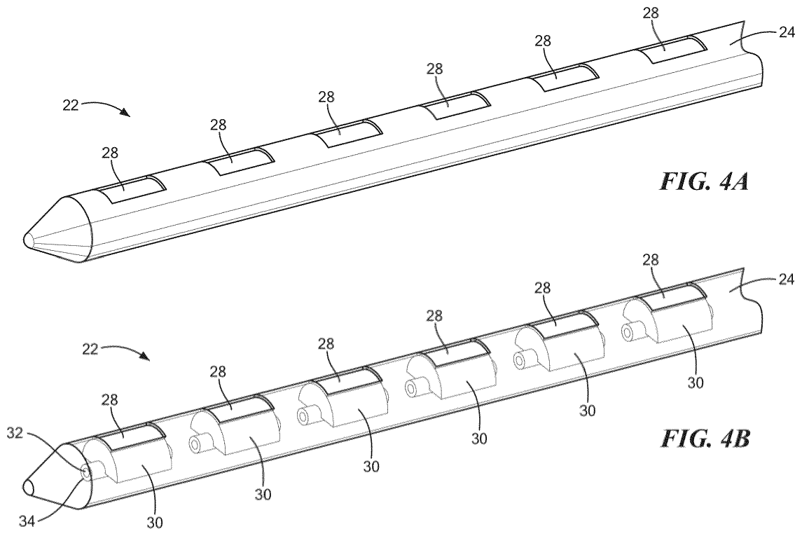

In other configurations, for example, as shown in FIG. 4, a plurality of expandable members 30 are included within the lumen 26 each in fluid communication with the supply lumen 32 and the exhaust lumen 34. Each of the expandable members 30 may define a shape that facilities the protrusion of the expandable member 30 from the corresponding slot 28. For example, each expandable member 30 may define a substantially semicylindrical shape such that when inflated with a cryogenic fluid, the expandable member 30 protrudes outward from its corresponding slot 28. In some configurations, the plurality of expandable members 30 are in fluid communication with a common supply lumen 32 and exhaust lumen 34. The exhaust lumen 34 is in fluid communication with an exhaust chamber 21 within the console 18. In other configurations, each expandable member 30 may be in communication with an independent supply line 32 and exhaust line 34 such that each expandable member 30 may be inflated and deflated independently of each and every other expandable member 30. A vacuum pump in the console 18 creates a low pressure environment in one or more lumens within the elongate shaft 24 so that refrigerant is drawn into the lumen(s), away from the expandable member 30, towards the proximal end portion 14 of the elongate shaft, and into the exhaust chamber 21 within the console 18.

As shown in FIG. 2, a stop 31 may be disposed within the lumen 26 proximal to the expandable member 30 within the shaft 24 or distal end portion 22. Additionally, although not shown in FIG. 3-5, it is to be understood that the stop 31 may also be disposed within the lumen 26 in these configurations. The stop 31 is configured to restrict longitudinal expansion of the expandable member 30 and to confine the expandable member 30 to a predefined space within the lumen 26. For example, the stop 31 may be a plug or other mechanical stop that is bonded or otherwise affixed within the lumen such that when the expandable member 30 expands it pushes on the stop 31 and is forced outward from the at least one slot 28 such that the expandable member 30 expands to a shape or configuration that conforms approximately to the length, size, and/or shape of the slot 28. As shown in FIGS. 1-4B, each slot 28 may be the same length, size, and/or shape as another slot 28. However, it is to be understood that each slot 28 may instead have a unique length, size, and/or shape that is different from an adjacent slot 28 or any other slot 28 defined within the shaft 24. In some configurations, the supply lumen 32 and the exhaust lumen 34 are tubes that extend through the stop 31 into the expandable member 30 for continuous flow of refrigerant into and out of the expandable member 30.

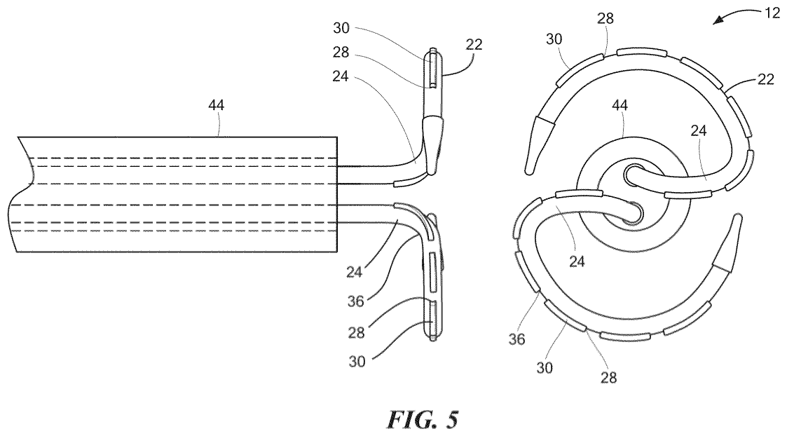

Referring now to FIG. 5, in some configurations, the medical device 12 can comprise one or more additional distal end portion(s) 36 in combination with distal end portion 22. The multiple distal end portions can be each attached to its own handle 16 and used together as part of the system, or the multiple distal end portion can have the elongate shaft 24 and a common handle between them. The additional distal end portion(s) 36 may be the same or similar in construction to the distal end portion 22 disclosed herein, therefore the same reference numerals are used to designate corresponding elements of the distal end portion 36 and a description of them and their operation is not repeated in the interest of brevity. In some configurations, for example as shown in FIG. 5, the distal end portion 22 and the distal end portion 36 may define spirals substantially in a single plane with contact surfaces arranged so as to treat circumferential section of the target tissue. In some configurations, when inflated, the distal end portions 22, 36, can be coplanar such that the distal end portions 22, 36, together define a group of contact surfaces for uniformly and/or symmetrically treating target tissue. In their deployed shapes, the distal end portions 22, 36, can separate from each other in different radial directions and then bend in the same circumferential direction, for example as shown in FIG. 5. In some configurations, the distal end portions 22, 36, can extend radially together or close to one another then bend in different circumferential directions. In some configurations, the distal end portions may be movable or slidable longitudinally or rotationally with respect to tissue or a guide catheter 44, if present, to provide for particular ablation patterns or contact surfaces.

In some configurations, the distal end portion 22 and the additional distal end portion(s) 36 of the medical device 12 disclosed herein may be delivered through a blood vessel to a target tissue region through a guide catheter 44 (for example, as shown in FIG. 5) or a guide wire, which is configured to be advanced through the blood vessels.

It will be appreciated by persons skilled in the art that the present invention is not limited to what has been particularly shown and described herein above. The features of various embodiments can be combined to form further embodiments of the invention that may not be explicitly described or illustrated. Each embodiment and each aspect so defined may be combined with any other embodiment or with any other aspect unless clearly indicated to the contrary. In addition, unless mention was made above to the contrary, it should be noted that all of the accompanying drawings are not to scale. A variety of modifications and variations are possible in light of the above teachings without departing from the scope and spirit of the invention, which is limited only by the following claims.

Aspects and embodiments of the invention may be defined by the following clauses.

Clause 1. A medical device, comprising:
an elongate shaft having a proximal end portion, a distal end portion, and defining a lumen therebetween;
the distal end portion defining at least one slot; and
an expandable member disposed within the lumen, the expandable member being configured to protrude outward from the at least one slot when expanded with cryogenic fluid.

Clause 2. The medical device of Clause 1, wherein the expandable member is an inflatable balloon.

Clause 3. The medical device of Clause 1, wherein the distal end portion is biased to a linear configuration.

Clause 4. The medical device of Clause 1, wherein the distal end portion is biased to a spiral configuration.

Clause 5. The medical device of Clause 1, wherein the at least one slot includes a plurality of slots, and wherein the plurality of slots is longitudinally spaced along the distal end portion.

Clause 6. The medical device of Clause 5, wherein the plurality of slots is disposed at a same radial position along the distal end portion.

Clause 7. The medical device of Clause 6, wherein each of the plurality of slots is a same size and evenly spaced from each other along the distal end portion.

Clause 8. The medical device of Clause 1, further comprising at least one additional distal end portion defining at least one slot through which the expandable member or an additional expandable member protrudes outward when expanded with cryogenic fluid.

Clause 9. The medical device of Clause 1, wherein the at least one slot is an elongated slot extending along a longitudinal portion of the distal end portion.

Clause 10. The medical device of Clause 1, further including a stop disposed within the lumen within the distal end portion, the stop being configured to restrict longitudinal expansion of the expandable member.

Clause 11. A medical device, comprising:
a first elongate shaft having a first proximal end portion and a first distal end portion and defining a first lumen therethrough;
the first distal end portion defining at least one first slot;
a first expandable member disposed within the first lumen, the first expandable member being configured to protrude outward from the at least one first slot when expanded;
a second elongate shaft having a second proximal end portion and a second distal end portion and defining a second lumen therethrough;
the second distal end portion defining at least one second slot;
a second expandable member disposed within the second lumen, the second expandable member being configured to protrude outward from the at least one second slot when expanded; and
the first and a second elongate shafts being disposed within a guide catheter.

Clause 12. The medical device of Clause 11, wherein the first and second expandable members are inflatable balloons.

Clause 13. The medical device of Clause 11, wherein the first and a second distal end portions are curved to define a spiral configuration.

Clause 14. The medical device of Clause 11, wherein the at least one first and second slots includes a plurality of first and second slots respectively, and wherein the plurality of first and second slots is longitudinally spaced along the respective first and second distal end portions.

Clause 15. The medical device of Clause 14, wherein the plurality of first and second slots is disposed at a same radial position along the respective first and a second distal end portions.

Clause 16. The medical device of Clause 15, wherein each of the plurality of first and second slots is a same size.

Clause 17. The medical device of Clause 16, wherein the plurality of first and second slots is evenly spaced along the respective first and second distal end portions.

Clause 18. The medical device of Clause 11, wherein the at least one first and second slots is an elongated slot extending along a longitudinal portion of the respective first and second distal end portions.

Clause 19. The medical device of Clause 11, further including a first stop disposed within the first lumen within the first distal end portion, the first stop being configured to restrict longitudinal expansion of the first expandable member.

Clause 20. A medical device, comprising:
an elongate shaft having a proximal end portion, a distal end portion, and defining a lumen therebetween;
the distal end portion defining a plurality of longitudinally and evenly spaced slots;
an inflatable balloon disposed within the lumen, the inflatable balloon being configured to protrude outward from the plurality of slots when inflated with a cryogenic fluid;
and a stop disposed within the lumen the stop being configured to restrict longitudinal inflation of the inflatable member.

1. A medical device, comprising:
an elongate shaft having a proximal end portion, a distal end portion, and defining a lumen therebetween;
the distal end portion defining at least one slot; and
an expandable member disposed within the lumen, the expandable member being configured to protrude outward from the at least one slot when expanded with cryogenic fluid.

2. The medical device of claim 1, wherein the expandable member is an inflatable balloon.

3. The medical device of claim 1, wherein the distal end portion is biased to a linear configuration.

4. The medical device of claim 1, wherein the distal end portion is biased to a spiral configuration.

5. The medical device of claim 1, wherein the at least one slot includes a plurality of slots, and wherein the plurality of slots is longitudinally spaced along the distal end portion.

6. The medical device of claim 5, wherein the plurality of slots is disposed at a same radial position along the distal end portion.

7. The medical device of claim 5, wherein each of the plurality of slots is a same size and evenly spaced from each other along the distal end portion.

8. The medical device of claim 1, further comprising at least one additional distal end portion defining at least one slot through which the expandable member or an additional expandable member protrudes outward when expanded with cryogenic fluid.

9. The medical device of claim 1, wherein the at least one slot is an elongated slot extending along a longitudinal portion of the distal end portion.

10. The medical device of claim 1, further including a stop disposed within the lumen within the distal end portion, the stop being configured to restrict longitudinal expansion of the expandable member.

11. A medical device, comprising:
a first elongate shaft having a first proximal end portion and a first distal end portion and defining a first lumen therethrough;
the first distal end portion defining at least one first slot;
a first expandable member disposed within the first lumen, the first expandable member being configured to protrude outward from the at least one first slot when expanded;
a second elongate shaft having a second proximal end portion and a second distal end portion and defining a second lumen therethrough;
the second distal end portion defining at least one second slot;
a second expandable member disposed within the second lumen, the second expandable member being configured to protrude outward from the at least one second slot when expanded; and
the first and a second elongate shafts being disposed within a guide catheter.

12. The medical device of claim 11, wherein the first and second expandable members are inflatable balloons.

13. The medical device of claim 11, wherein the first and a second distal end portions are curved to define a spiral configuration.

14. The medical device of claim 11, wherein the at least one first and second slots includes a plurality of first and second slots respectively, and wherein the plurality of first and second slots is longitudinally spaced along the respective first and second distal end portions.

15. The medical device of claim 14, wherein the plurality of first and second slots is disposed at a same radial position along the respective first and a second distal end portions.

16. The medical device of claim 14, wherein each of the plurality of first and second slots is a same size.

17. The medical device of claim 14, wherein the plurality of first and second slots is evenly spaced along the respective first and second distal end portions.

18. The medical device of claim 11, wherein the at least one first and second slots is an elongated slot extending along a longitudinal portion of the respective first and second distal end portions.

19. The medical device of claim 11, further including a first stop disposed within the first lumen within the first distal end portion, the first stop being configured to restrict longitudinal expansion of the first expandable member.

20. A medical device, comprising:
an elongate shaft having a proximal end portion, a distal end portion, and defining a lumen therebetween;
the distal end portion defining a plurality of longitudinally and evenly spaced slots;
an inflatable balloon disposed within the lumen, the inflatable balloon being configured to protrude outward from the plurality of slots when inflated with a cryogenic fluid; and
a stop disposed within the lumen the stop being configured to restrict longitudinal inflation of the inflatable member.
